(19) United States
(12) Patent Application Publication
(10) Pub. No.: US 2003/0078559 A1
(43) Pub. Date: Apr. 24, 2003

(54) Collection bag with insert

(76) Inventors: Francis Goudaliez, Faches-Thumesnil (FR); Thierry Verpoort, Mouvaux (FR)

Correspondence Address: Baker & Botts, One Shell Plaza, 910 Louisiana Street, Houston, TX, 77002-4995, US

(21) Appl. No.: 10/239,624

(22) PCT Filed: Mar. 21, 2001
(86) PCT No.: PCT/FR01/00851

(30) Foreign Application Priority Data
Mar. 24, 2000 (FR) 00/03802

(51) Int. Cl. A61B 19/00

(52) U.S. Cl. 604/410

(57) ABSTRACT

The invention concerns a collection bag (1) for a fluid, in particular whole blood, comprising an outer envelope (2) formed with two sheets of flexible plastic material (3, 4) assembled on their periphery so as to define an inner volume, said outer envelope (2) being provided with at least an orifice designed to enable the fluid to be supplied and dispensed, wherein at least one insert (15) is fixed in the internal volume of the bag (1) to define at least two sub-volumes (16, 17) in the internal volume, said insert (15) being arranged to form a partial obstruction to the flow of the fluid inside said internal volume leaving at least a passage for the fluid (19, 20, 22, 24) between said two sub-volumes (16, 17).

The invention relates to a bag for collecting a fluid, a bag system comprising at least one such bag, and the use thereof.

It is applicable typically where whole blood is taken from a donor in order to be collected in a collection bag, the whole blood then being capable of being treated in a bag system, in particular by centrifuging and/or filtration, so as to separate the blood into its different constituents.

Collection bags are already known comprising an outer envelope formed from two sheets of flexible plastic material assembled on their periphery so as to define an internal volume, said outer envelope being provided with at least one orifice arranged to allow the supply and dispensing of the fluid or its components.

In order to take whole blood from a donor, a tube is associated on the one hand with an input orifice of the collection bag and on the other hand with means of taking the blood, for example formed from a needle, which are inserted into a vein of the donor.

In order to increase the keeping time of the blood, it is conventional for the collection bag to be filled, prior to the blood being taken, with an anticoagulant and/or blood preservative solution.

One of the problems which then arises is to achieve, as the blood arrives in the collection bag, a homogeneous mixture between the solution contained in the bag and the blood.

In order to solve this problem, the blood and the solution are mixed while the blood is being taken, conventionally by agitation of the bag. The aim of the mixing is in particular to make Ca²⁺ ions of the blood come together with the citrate ions of the anticoagulant and/or preservative solution.

However, tests show that, under these conditions, coagulation of the blood is not prevented satisfactorily, for example with a view to filtration of the whole blood. This is because the agitation forces are not sufficient to provide homogeneous mixing between the solution and the blood which are different in their density, temperature and volume. Moreover, with the agitation intensity being constant during taking of the blood, the more the bag fills up, the less the agitation creates turbulence favourable to the mixing of the bag contents.

In particular, this poor homogenisation involves the formation of blood clots which are detrimental from the viewpoint of filtration of the blood or its components since they have a tendency to clog the filter medium.

But the formation of fibrin is also detrimental to the quality of the plasma through loss of some of the factors involved in coagulation.

The invention therefore aims to remedy these drawbacks by proposing a collection bag which makes it possible to reduce the formation of blood clots, in particular by improving the implementation of the mixing between the anticoagulant and/or preservative solution and the blood that has been taken.

To that end, and according to a first aspect, the invention proposes a bag for collecting a fluid, in particular whole blood, of the type comprising an outer envelope formed from two sheets of flexible plastic material assembled on their periphery so as to define an internal volume, said outer envelope being provided with at least one orifice arranged to allow the supply and dispensing of the fluid, in which at least one insert is fixed in the internal volume of the bag so as to define on either side of said insert at least two sub-volumes in the internal volume, said insert being arranged to form a partial obstacle to the flow of the fluid inside said internal volume while leaving at least one passage for fluid between said sub-volumes.

In a variant, the bag contains an anticoagulant and/or preservative solution for the fluid intended to be collected.

According to a first embodiment, the insert is formed from a strip of material.

According to a second embodiment, the insert is formed from two strips of material associated with one another so as to leave a passage for fluid between them.

According to a third embodiment, the insert is formed from a plurality of strips of material associated in the form of a grid so as to define a multitude of passages for fluid.

In a variant, the strips of material are formed from flexible plastic, in particular the same as that forming the outer envelope.

According to one implementation, the insert is disposed substantially transversely by being fixed in the vicinity of the area of assembly of the two sheets.

According to a second aspect, the invention proposes a bag system comprising at least one collection bag as described above, in which the collection bag is connected, by means of an input orifice, to means of taking the fluid and, by means of an output orifice, to a filtration unit or to one or more other bags.

In a variant, the bag system comprises, associated downstream of the filtration unit, a set of bags allowing separation of the blood into its different constituents.

In a variant, one of the bags of the set is a collection bag as described above.

According to a third aspect, the invention relates to the use of a collection bag as described above or of a bag system as described above, in which the collection bag is agitated so that the insert can contribute towards homogenisation of the fluid contained in the bag.

Other objects and advantages of the invention will emerge during the description that follows with reference to the accompanying drawings.

FIG. 1 depicts a flat front view of a first embodiment of a collection bag according to the invention.

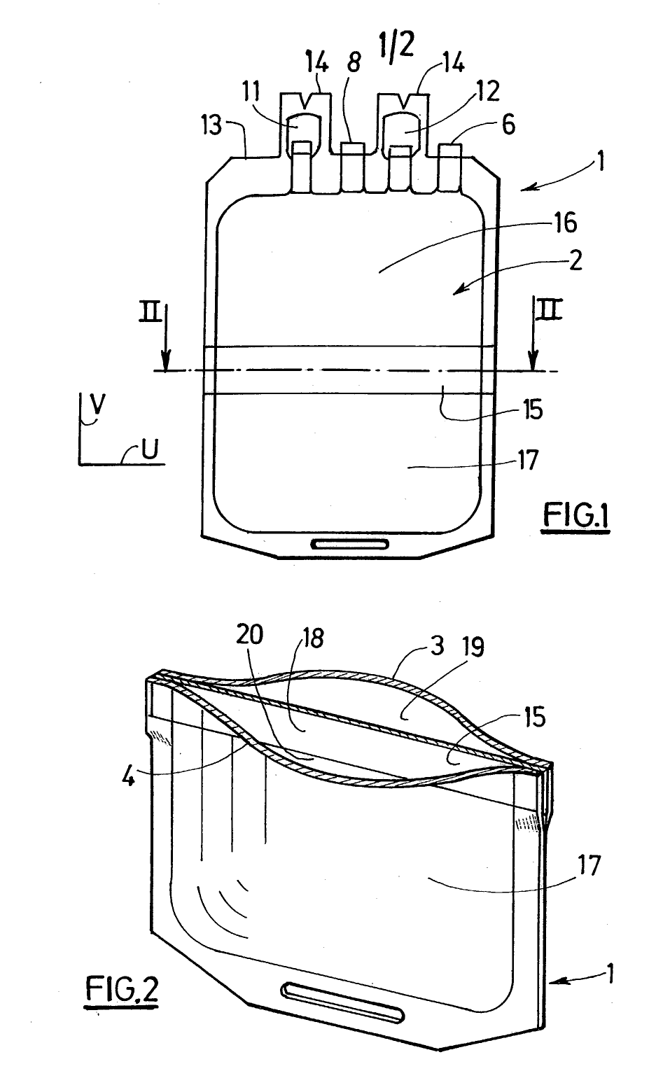

FIG. 2 is a perspective view in cross-section along the line II-II of FIG. 1.

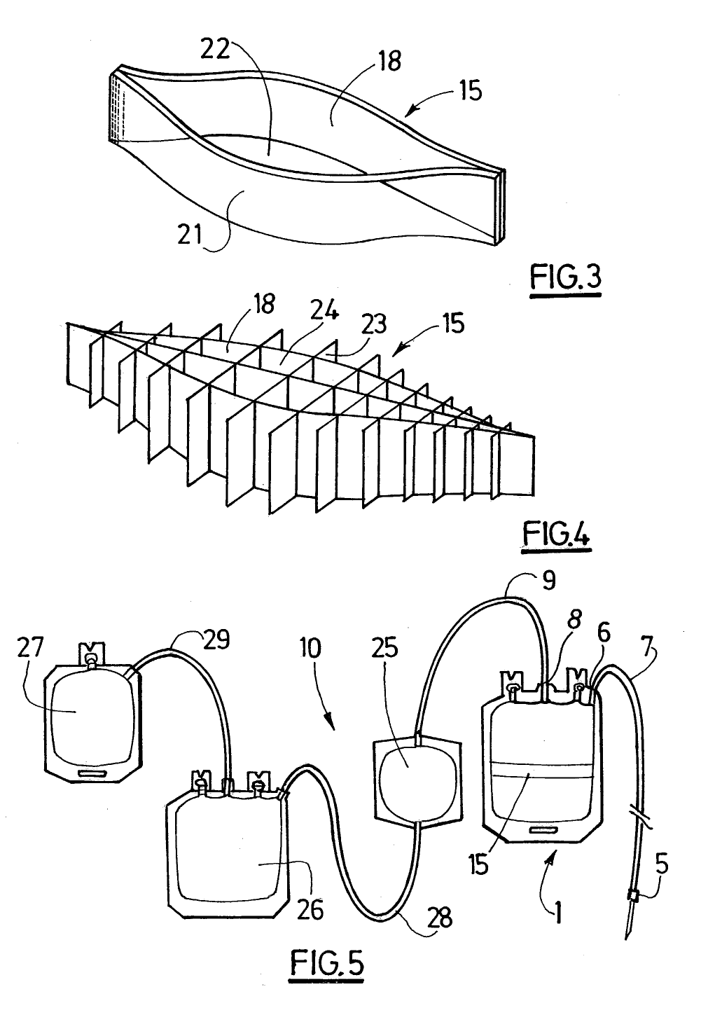

FIG. 3 is a perspective depiction of a second embodiment of an insert intended to be inserted into a collection bag.

FIG. 4 is a perspective depiction of a third embodiment of an insert intended to be inserted into a collection bag.

FIG. 5 is a schematic flat front view of a bag system comprising a collection bag according to the invention.

In the particular implementation depicted in particular in FIG. 1, a fluid collection bag 1 comprises an outer envelope 2 formed from two sheets 3, 4 of plastic material assembled, for example by welding, on their periphery so as to define an internal volume.

The sheets 3, 4 are formed from a biocompatible flexible plastic material which is weldable and sterilisable, for example polyvinyl chloride.

Such a collection bag 1 is in particular intended to collect whole blood taken from a donor using taking means 5, for example formed from a needle.

To that end, the outer envelope 2 is provided with an input orifice 6 intended to receive, so as to be impervious, the upstream part of a tube 7 having at its downstream end the taking means 5. This implementation makes it possible to put into fluidic communication the taking means 5 and the internal volume of the bag 1 so as to be able to supply the bag 1 with fluid.

An output orifice 8 is also arranged on the outer envelope 2 in order to allow dispensing of the fluid. A tube 9 can then be associated with the output orifice 8 so as to be able to supply with fluid a bag system 10 such as, for example, that described in the remainder of this description.

In the embodiments depicted in the figures, the two orifices 6, 8 are disposed on the same side of the envelope 2, but they can be disposed differently, for example on two opposite or adjacent sides of the envelope 2. Similarly, a single orifice only 6, 8 can be provided used for both supplying and dispensing the fluid.

In the description, the terms “upstream” and “downstream” are defined respectively with respect to the direction of movement of the fluid from the taking means 5 to the output orifice 8, via the collection bag 1.

In the embodiment depicted in FIG. 1, two other orifices 11, 12 are disposed conventionally on the same side 13 of the envelope 2 as the input orifice 6 and output orifice 8. The two orifices 11, 12 are sealed so as to be impervious and sterile by a device 14 that can be torn away should the operator wish to use them as a second input and/or output orifice.

The collection bag 1 that has just been described constitutes a unitary assembly that can be incorporated into a more complex system that comprises for example other bags, tubes, clamps or filters. An example of such a bag system 10 is described subsequently.

According to the invention, at least one insert 15 is fixed in the internal volume of the bag 1 so as to define on either side of said insert at least two sub-volumes 16, 17 in the internal volume, said insert 15 being arranged to form a partial obstacle to the flow of the fluid inside said internal volume while leaving at least one passage for fluid between said sub-volumes 16, 17.

The fluid can then communicate freely between the sub-volumes 16, 17, in particular the insert 15 has no filtering function but has a function similar to that of a stirring blade when the bag 1 is agitated.

The insert is intended to further the mixing between the fluid and an anticoagulant and/or preservative solution when the bag 1 is subjected to appropriate movements such as those created during agitation of the bag.

In one particular example, the solution is introduced into the bag 1 prior to it being supplied with fluid but it can be introduced, by means of an input orifice of the bag 1, substantially at the same time as or after the fluid.

There will now be described a first, a second and then a third embodiment of the insert 15.

In the first embodiment (FIGS. 1 and 2), the insert 15 is formed from a strip of material 18 made from a flexible plastic material which is welded between the two sheets 3, 4 in the vicinity of their area of assembly. In one particular example, the materials forming the insert 1 and the sheets 3, 4 are identical so as to facilitate the welding.

The insert 15 is disposed substantially transversely (in the direction U shown in FIG. 1) with respect to the bag 1 so as to define a first sub-volume 16 disposed on the side of the input orifice 6 and output orifice 8 and a second sub-volume 17 opposite the first 16 with respect to the insert 15.

The two sub-volumes 16, 17 are then defined on either side of the insert 15 and are delimited by the corresponding parts of the outer envelope 2.

As illustrated more particularly in FIG. 2, the insert 15 leaves two passages for fluid 19, 20 between these two sub-volumes 16, 17. The first 19 is defined between the first sheet 3 and the insert 15, and the second 20 between the insert 15 and the second sheet 4.

During agitation of the bag 1, the fluid then flows from the first sub-volume 16 to the second 17, and vice versa, the insert 15 forming a partial obstacle to this flow.

In the second embodiment (FIG. 3), a second strip of material 21, identical or not to the first 18, is disposed adjacent to that described in connection with the first embodiment, so as to form an insert 15.

As in the previous embodiment, the insert 15 is welded so as to be impervious between the two sheets 3, 4 of the bag 1. In a variant, the implementation of this welding is carried out so as to associate the two sheets 3, 4 and the two strips of material 18, 20 in a single step.

In this embodiment, the two sub-volumes 16, 17 are the same as those defined previously, that is to say distributed either side of the insert 15 in the direction V. The insert 15 then leaves three passages for fluid between these two sub-volumes 16, 17, the first 19 between the sheet 3 and the first strip 18, the second 22 between the two strips 18, 21 and the third 20 between the second strip 21 and the sheet 4.

In the third embodiment (FIG. 4), the insert 15 is formed from a plurality of strips of material 18, 23 associated in the form of a grid so as to define a multitude of passages 24 for fluid.

The insert 15 depicted in FIG. 4 comprises three strips of material 18 as defined in the first embodiment and a number of strips of material 23 disposed substantially perpendicular to these three strips 18 and spaced apart from one another. The association of this set of strips of material 18, 23 is carried out, for example by welding, prior to the fixing of the insert 15 in the bag 1.

As in the previous embodiments, the insert 15 is welded substantially transversely between the two plastic sheets 3, 4 of the bag 1 so as to leave a plurality of passages for fluid 24 between the two sub-volumes 16, 17 defined above.

In the three embodiments described, the bag 1 comprises a single insert 15 but a number of inserts 15, identical or different, can be provided, disposed inside the bag 1, so as to further improve the implementation of the mixing between the anticoagulant and/or preservative solution and the fluid that has been taken.

There will now be described, in connection with FIG. 5, a bag system 10 comprising a collection bag 1 inside which an insert 15 is fixed.

The input orifice 6 is connected, by means of a first tube 7, to means of taking the fluid 5.

The output orifice 8 is connected, by means of a second tube 9, to a filtration unit 25 capable in particular of removing leukocytes from the whole blood.

Downstream of the filtration unit 25, a first 26 and a second 27 satellite bag are connected in series by means respectively of a third 28 and a fourth 29 tube.

Such a bag system 10 makes it possible, after collection of the whole blood, to carry out in closed circuit the filtration thereof so as to collect in the first satellite bag 26 whole blood with the leukocytes removed. The first satellite bag 26 can then be centrifuged so as for example to obtain, after transfer, a red cell concentrate in the first satellite bag 26 and blood plasma in the second satellite bag 27.

According to one embodiment, the tubes 7, 9, 28 and 29 are divisible and weldable so as to make it possible, prior to the centrifuging of the bag 26, to separate the filter 25 and the bag 1 of the system 10 by cutting and welding of the tube 28.

In a variant, an insert 15 is fixed inside the bag 26. In this implementation, a preservative solution can be introduced into the bag 26 after the transfer of the plasma into the bag 27. This introduction can be carried out in closed circuit by making provision that the system 10 also comprises a bag containing the preservative solution which is in fluidic communication with the bag 26.

The use of a collection bag 1 or of a bag system 10 is described below briefly.

The collection bag 1 contains an anticoagulant and/or preservative solution for the fluid intended to be collected, for example of CPD type for the collection of whole blood or SAG-Mannitol for that of a red cell concentrate.

While blood is being taken from the donor, the bag 1 is agitated so as to mix said solution with the blood, as soon as the bag 1 is started to be supplied with blood.

In one embodiment, the agitation is performed by partial rotation of the bag 1 around the axis U shown in FIG. 1. The passages for fluid 19, 20, 22, 24 left by the insert then extend substantially perpendicular to the direction U (direction V shown in FIG. 1).

The presence of the insert 15 inside the collection bag 1 then allows better mixing of the blood and the solution during agitation since the fluid flows alternately from the first 16 to the second 17 sub-volume by means of the passages for fluid 19, 20, 22, 24. The insert 15 then forms a partial obstacle to this flow so as to improve the homogenisation of the mixture by causing turbulence.

In the absence of an anticoagulant and/or preservative solution prior to the introduction of the fluid into the collection bag 1, the insert 15 allows the agitation of the fluid to be improved by forming turbulence in the flow, which reduces the phenomenon of blood coagulation. The anticoagulant and/or preservative solution is introduced, during agitation of the bag, substantially at the same time as or after the fluid.

In the three embodiments described, the insert 15 is disposed substantially transversely inside the bag 1. Another disposition can however be envisaged, in particular in the case of another mode of agitation of the bag 1.

By virtue of the invention, the conditions for preserving the blood are improved in that in particular the formation of blood clots in the collected whole blood is reduced.

The whole blood can then be filtered, in particular in closed circuit in a system 10 such as that described previously, in a more satisfactory manner by reducing the risks of clogging of the filter medium by the blood clots.

To that end, the collection bag 1 can be agitated, not only while the blood is being taken but also before filtration so as to benefit fully from the advantages provided by the invention.

Where an insert 15 is fixed inside the bag 26, the preservative solution is introduced into the bag 26 after centrifuging and transfer of the plasma into the bag 27 and then the bag 26 is agitated, in particular manually by the operator, so as to improve the mixing between the red cell concentrate and the preservative solution.

1. A bag for collecting (1) a fluid, in particular whole blood, of the type comprising an outer envelope (2) formed from two sheets of flexible plastic material (3, 4) assembled on their periphery so as to define an internal volume, said outer envelope (2) being provided with at least one orifice (6, 8) arranged to allow the supply and dispensing of the fluid, characterised in that at least one insert (15) is fixed in the internal volume of the bag (1) so as to define on either side of said insert (15) at least two sub-volumes (16, 17) in the internal volume, said insert (15) being arranged to form a partial obstacle to the flow of the fluid inside said internal volume while leaving at least one passage for fluid (19, 20, 22, 24) between said sub-volumes (16, 17).

2. A bag according to claim 1, characterised in that it contains an anticoagulant and/or preservative solution for the fluid intended to be collected.

3. A bag according to claim 1 or 2, characterised in that the insert (15) is formed from at least one strip of material.

4. A bag according to claim 3, characterised in that the insert (15) is formed from two strips of material (18, 21) associated with one another so as to leave a passage for fluid (22) between them.

5. A bag according to claim 3, characterised in that the insert (15) is formed from a plurality of strips of material (18, 23) associated in the form of a grid so as to define a multitude of passages (24) for fluid.

6. A bag according to any one of claims 3 to 5, characterised in that the strips of material (18, 21, 23) are formed from flexible plastic, in particular the same as that forming the outer envelope (2).

7. A bag according to any one of claims 1 to 6, characterised in that the insert (15) is disposed substantially transversely by being fixed in the vicinity of the area of assembly of the two sheets (3, 4).

8. A bag system (10) comprising at least one collection bag (1) according to any one of claims 1 to 7, characterised in that the collection bag (1) is connected, by means of an input orifice (6), to means of taking the fluid (5) and, by means of an output orifice (8), to a filtration unit (25) or to one or more other bags.

9. A bag system according to claim 8, characterised in that it comprises, associated downstream of the filtration unit (25), a set of bags (26, 27) allowing separation of the blood into its different constituents.

10. A bag system according to claim 9, characterised in that the bag 26 is a collection bag according to any one of claims 1 to 7.

11. Use of a bag (1) according to any one of claims 1 to 7 or of a bag system (10) according to any one of claims 8 to 10, in which the collection bag (1, 26) is agitated so that the insert (15) can contribute towards homogenisation of the fluid contained in said bag (1, 26).
